(19) United States
(12) Patent Application Publication
(10) Pub. No.: US 2005/0053949 A1
(43) Pub. Date: Mar. 10, 2005

(54) Biochip for proteomics applications

(76) Inventor: Vitalii Ivanovich Silin, Germantown, MD (US)

Correspondence Address: Vitalii Silin, 12415 Milestone Manor Lane, Germantown, MD, 20876, US

(21) Appl. No.: 10/656,369

(22) Filed: Sep. 8, 2003

(51) Int. Cl. C12Q 1/68

(52) U.S. Cl. 435/6

(57) ABSTRACT

Arrays of binding molecules/instigators useful for the simultaneous detection of a plurality of proteins, which are the expression products, or fragments thereof, of a cell, or population of cells in an organism are provided. Methods of both making and using the arrays are also disclosed. The arrays are particularly useful for various proteomics applications including assessing patterns of protein expression and modification in cells and can be useful for biowarfare agents detection.

This application claims priority of provisional patent application with registration # 38,435 filed on Sep. 10, 2002 and title: “Biochip for proteomics applications”

References Cited
U.S. Patent Documents

| | | | |
|---|---|---|---|
| 4,071,409 | January, 1978 | Messing et al. | 195/63. |
| 4,562,157 | December, 1985 | Lowe et al. | 435/291. |
| 4,690,715 | September, 1987 | Allara et al. | 148/6. |
| 4,908,112 | March, 1990 | Pace | 204/299. |
| 4,973,493 | November, 1990 | Guire | 427/2. |
| 4,987,032 | January, 1991 | Miyasaka et al. | |
| 5,079,600 | January, 1992 | Schnur et al. | 357/4. |
| 5,096,807 | March, 1992 | Leaback | 435/6. |
| 5,143,854 | September, 1992 | Pirrung et al. | 438/518. |
| 5,154,808 | October, 1992 | Miyasaka et al. | |
| 5,160,597 | November, 1992 | Colapicchioni et al. | 204/403. |
| 5,242,828 | September, 1993 | Bergstrom et al. | 435/291. |
| 5,252,743 | October, 1993 | Barrett et al. | 548/303. |
| 5,294,369 | March, 1994 | Shigekawa et al. | 252/313. |
| 5,304,487 | April, 1994 | Wilding et al. | |
| 5,342,692 | August, 1994 | Ribi | 428/420. |
| 5,384,261 | January, 1995 | Winkler et al. | 436/518. |
| 5,405,766 | April, 1995 | Kallury et al. | |
| 5,405,783 | April, 1995 | Pirrung et al. | 436/518. |
| 5,412,087 | May, 1995 | McGall et al. | 536/24. |
| 5,429,708 | July, 1995 | Linford et al. | 216/66. |
| 5,445,934 | August, 1995 | Fodor et al. | 435/6. |
| 5,472,881 | December, 1995 | Beebe et al. | 436/94. |
| 5,510,270 | April, 1996 | Fodor et al. | 436/518. |
| 5,512,131 | April, 1996 | Kumar et al. | 156/655. |
| 5,512,492 | April, 1996 | Herron et al. | 436/518. |
| 5,514,501 | May, 1996 | Tarlov | 430/5. |
| 5,520,787 | May, 1996 | Hanagan et al. | 204/409. |
| 5,571,410 | November, 1996 | Swedberg et al. | 210/198. |
| 5,593,838 | January, 1997 | Zanzucchi et al. | 435/6. |
| 5,603,351 | February, 1997 | Cherukuri et al. | 137/597. |
| 5,605,662 | February, 1997 | Heller et al. | 422/68. |
| 5,620,850 | April, 1997 | Bamdad et al. | 530/300. |
| 5,622,826 | April, 1997 | Varma | 435/6. |
| 5,624,711 | April, 1997 | Sundberg et al. | 427/261. |
| 5,629,213 | May, 1997 | Kornguth et al. | 436/518. |
| 5,635,358 | June, 1997 | Wilding et al. | 435/7. |
| 5,637,469 | June, 1997 | Wilding et al. | 435/7. |
| 5,677,195 | October, 1997 | Winkler et al. | 436/518. |
| 5,677,196 | October, 1997 | Herron et al. | 436/518. |
| 5,681,484 | October, 1997 | Zanzucchi et al. | 216/2. |
| 5,688,642 | November, 1997 | Chrisey et al. | 435/6. |
| 5,700,642 | December, 1997 | Monforte et al. | 435/6. |
| 5,719,060 | February, 1998 | Hutchens et al. | 436/174. |
| 5,726,026 | March, 1998 | Wilding et al. | |
| 5,731,152 | March, 1998 | Maracas et al. | 3/98. |
| 5,744,305 | April, 1998 | Fodor et al. | 435/6. |
| 5,776,674 | July, 1998 | Ulmer. | |
| 5,837,832 | November, 1998 | Chee et al. | 536/22. |
| 5,861,242 | January, 1999 | Chee et al. | 435/5. |
| 5,866,345 | February, 1999 | Wilding et al. | |
| 5,925,552 | July, 1999 | Keogh et al. | |
| 5,928,880 | July, 1999 | Wilding et al. | |
| 6,329,209 | December 2001 | Wagner et al. | 436/518 |
| 6,365,418 | April 2002 | Wagner et al. | 436/518 |

Foreign Patent Documents

| | | |
|---|---|---|
| WO9850773 | November, 1994 | WO. |
| WO9535505 | December, 1995 | WO. |
| WO 96/02830 | February, 1996 | WO. |
| WO 96/10178 | April, 1996 | WO. |
| WO 96/26432 | August, 1996 | WO. |
| WO 96/29629 | September, 1996 | WO. |
| WO 96/38726 | December, 1996 | WO. |
| WO 96/39937 | December, 1996 | WO. |
| WO 97/07429 | February, 1997 | WO. |
| WO 97/36681 | October, 1997 | WO. |
| WO 97/41424 | November, 1997 | WO. |
| WO 97/41425 | November, 1997 | WO. |
| WO9823948 | June, 1998 | WO. |
| WO9839481 | September, 1998 | WO. |

### OTHER REFERENCES

Sigal et al. (1996). A self-assembled monolayer for the binding and study of histidine-tagged proteins by surface plasmon resonance. Anal. Chem. 68:490-497.*
Nock et al. (1997). Reversible, site-specific immobilzation of polyarginine-tagged fusion proteins on mica surfaces. FEBS Letters. 414:233-238.*
Marks et al. (1991). By-passing immunization—Human antibodies from V-gene libraries displayed on phage. J. Mol. Biol. 222:581-597.*
Rowe et al. (1999). Array biosensor for simultaneous identification of bacterial, viral and protein analytes. Anal. Chem. 71(17):3846-3852.*
Dzgoev et al. (1996). Microformat imaging ELISA for pesticide determination. Anal. Chem. 68(19):3364-3369.*
Jones et al. (1998). Microminiaturized immunoassays using atomic force microscopy and compositionally patterned antigen arrays. Anal. Chem. 70(7):1223-1241.*
Collioud et al. (1993). Oriented and covalent immobilization of target molecules to solid supports: Synthesis and application of a light-activatable and thiol-reactive cross-linking reagent. Bioconjugate Chem. 4:528-536.*
Elkins et al. (1991). Multianalyte microspot immunoassay-microanalytical “compact disk” of the future. Clin. Chem. 37(11):1955-1967.*
Kricka (1998). Miniaturization of analytical systems. Clin. Chem. 44(9):2008-2014.*
Ekins (1998). Ligand assays: from electrophoresis to miniaturized microarrays. Clin. Chem. 44(9):2015-2030.*
Silzel et al. (1998). Mass-sensing, multianalyte microarray immunoassay with imaging detection. Clin. Chem. 44(9):2036-2043.*
Niemeyer et al. (1994). Oligonucleotide-directed self-assembly of proteins: semisyntetic DNA-streptavidin hybrid molecules as connectors for the generating of macroscopic arrays and the construction of supra-molecular bio-conjugates. Nucleic Acid Research 22(25): 5530-5539.*
Niemeyer et al. (1999). DNA-directed immobilization: efficient, reversible, and site-selective surface binding of proteins by means of covalent DNA-streptavidin conjugates. Anal. Biochemistry 268:54-63.*
Niemeyer et al. (1999). Functionalization of covalent DNA-streptavidin conjugates by means of biotinilated modulator components. Bioconjugate Chem. 10:708-719.*
Niemeyer et al. Self-assembly of DNA-streptavidin nanostructures and their use as reagents in immuno-PCR. Nucleic Acid Research 27(23):4553-4561 (1999).
Niemeyer et al. Hybritization characteristics of biomolecular adaptors, covalent DNA-streptavidin conjugates. Bioconjugate Chem. 9:168-175 (1998).
Wang et al. Peptide nucleic acid probes for sequence-specific DNA biosensors. J.Am.Chem.Soc. 118:7667-7670(1996).
Nielsen P. E. Application of peptide nucleic acids. Current Opinions in biotechnology, 10:71-75 (1999).
Arlinghaus et al. Analysis of biosensor chips for identification of nucleic acids. Anal.Chem. 69:3747-3753 (1997).
Lemmo et al.: “Characterization of inkjet chemical microdispensor for combinatorial library synthesis”, Anal. Chem. 69: 543-551 (1997).
Steinnicke et al. “Biochemical characteristics of caspases-3, -6, -7, and -8” J. Biol. Chem. 272: 25719-25723 (1997).
Talanian et al. “Substrate specialities of capsases family proteases” J. Biol. Chem. 272: 9677-9682 (1997).
Thornberry: “Interleukin-1beta converting enzyme” Meth. Enzymology 244: 615-631 (1994).
Villa et al. “Caspases and caspase inhibitors” TIBS 22: 388-393 (1997).
Kemeny: “Enzyme-linked immunoassays” in Immuno Chemistry 1 (eds Johnstone and Turner) p 147-175 (November 1997).
Mauracher et al. “Reduction of rubella ELISA background using heat denatured sample buffer” J. Immun. Meth. 145:251-254 (1991).
Pale-Grosdemange et al.: “Formation of self-assembled monolayers by chemisorption of derivatives of oligo(ethylene glycol) of structure HS(CH2) 11 (OCH2CH2)mOH on gold” J. Am. Chem. Soc. 113(1)12-20 (1991).
Pham, et al. “Human Interleukin-2 Production in Insect (Trichoplusia ni) Larvae: Effects and Partial Control of Proteolysis”, Biotechnology and Bioengineering vol. 62(2) pp. 175-182, Jan. 20, 1999.
Becker et al., “Fabrication of microstructures with high aspect ratios and great structural heights by synchrotron radiation lithography, galvanoforming, and plastic moulding (LIGA process),” Microelectronic Engineering, 4:35-36 (1986).
Becker et al., “Production of separation-nozzle systems for uranium enrichment by a combination of x-ray lithography and galvanoplastics,” Naturwissenschaften, 69:520-523 (1982).
Cload et al., “Development of improved tRNAs for in vitro biosynthesis of proteins containing unnatural amino acids,” Chemistry & Biology 3:1033-1038 (1996).
Condra et al., “In vivo emergence of HIV-1 variants resistant to multiple protease inhibitors,” Nature, 374:569-571 (1995).
Dammer et al., “Specific antigen/antibody interactions measured by force microscopy,” Biophysical Journal, 70:2437-2441 (1996).
Dawson et al., “Peptide-derived self-assembled monolayers: adsorption of N-stearoyl I-Cysteine Methyl Ester on Gold,” Journal of Molecular Recognition, 10:18-25 (1997).
Duschl et al., “Surface engineering: optimization of antigen presentation in self-assembled monolayers,” Biophysical Journal, 70:1985-1995 (1996).
Ellman et al., “Biosynthetic method for introducing unnatural amino acids site-specifically into proteins,” Methods in Enzymology, 202:301-336 (1991).
Geoghegan et al., “Fluorescence-based continuous assay for the aspartyl protease of human immunodeficiency virus-1,” FEBS, 262:119-122 (1990).
Hegner et al., “Ultralarge atomically flat template-stripped Au surfaces for scanning probe microscopy,” Surface Science, 291:39-46 (1993).
Hegner et al., “Immobilizing DNA on gold via thiol modification for atomic force microscopy imaging in buffer solutions,” FEBS, 336(3):452-456 (1993).
Hegner et al., “Modified DNA immobilized on bioreactive self-assembled monolayer on gold for dynamic force microscopy imaging in aqueous buffer solution,” J. Vac. Sci. Technol. B, 14(2):1418-1421 (1996).
Ho et al., “Characterization of human immunodeficiency virus type 1 variants with increased resistance to a C.sub.2 -symmetric protease inhibitor,” Journal of Virology, 68:2016-2020 (1994).
Hochuli et al., “Genetic approach to facilitate purification of recombinant proteins with a novel metal chelate adsorbent,” Biotechnology, 6:1321-1325 (1988).
Kaplan et al., “Selection of multiple human immunodeficiency virus type 1 variants that encode viral proteases with decreased sensitivity to an inhibitor of the viral protease,” Proc. Natl. Acad. Sci. USA, 91:5597-5601 (1994).
Korant et al., “The HIV protease and therapies for aids,” Advances in Experimental Medicine and Biology, 421:279-284 (1997).
Linford et al., “Alkyl monolayers on silicon prepared from 1-alkenes and hydrogen-terminated silicon,” J. Am. Chem. Soc., 117:3145-3155 (1995).
Loeb et al., “Complete mutagenesis of the HIV-1 protease,” Nature, 340:397-400 (1989).
Louis et al., “Autoprocessing of the HIV-1 protease using purified wild-type and mutated fusion proteins expressed at high levels in Escherichia coli,” Eur. J. Biochem., 199:361-369 (1991).
Mrksich et al., “Controlling cell attachment on contoured surfaces with self-assembled monolayers of alkanethiolates on gold,” Proc. Natl. Acad. Sci. USA, 93:10775-10778 (1996).
Moore et al., “Peptide substrates and inhibitors of the HIV-1 protease,” Biochemical and Biophysical Research Communications, 159:420425 (1989).
Nock, “Reversible, site-specific immobilization of polyarginine-tagged fusio proteins on mica surfaces,” FEBS, 414:233-238 (1997).
Noren et al., “A general method for site-specific incorporation of unnatural amino acids into proteins,” Science, 244:182-188 (1989).
Prime et al., “Self-assembled organic monolayers: model systems for studying absorption of proteins at surfaces,” Science, 252:1164-1167 (1991).
Roberts et al., “Rational design of peptide-based HIV proteinase inhibitors,” Science, 248:358-361 (1990).
Schock et al., “Mutational anatomy of an HIV-1 protease variant conferring cross-resistance to protease inhibitors in clinical trials,” The Journal of Biological Chemistry, 271:31957-31963 (1996).
Singhvi et al., “Engineering cell shape and function,” Science, 264:696-698 (1994).
Skalka, “Retroviral proteases: first glimpses at the anatomy of a processing machine,” Cell, 56:911-913 (1989).
Sundberg et al., “Spatially-addressable immobilization of macromolecules on solid supports,” J. Am. Chem. Soc. 117:12050-12057 (1995).
Wagner et al., “.omega.-functionalized self-assembled monolayers chemisorbed on ultraflat Au(111) surfaces for biological scanning probe microscopy in aqueous buffers,” J. Vac. Sci. Technol. B, 14(2):1466-1471 (1996).
Wagner et al., “Formation and in Situ modification of monolayers chemisorbed on ultraflat template-stripped gold surfaces,” Langmuir, 11(10):3867-3875 (1995).
Wagner et al., “Bioreactive self-assembled monolayers on hydrogen-passivated Si(111) as a new class of atomically flat substrates for biological scanning probe microscopy,” Journal of Structural Biology, 119:189-201 (1997).
Wagner et al., “Covalent immobilization of native biomolecules onto Au(111) via N-hydroxysuccinimide ester functionalized self-assembled monolayers for scanning probe microscopy,” Biophysical Journal, 70:2052-2066 (1996).
Weiner et al., “Site-directed mutagenesis of double-stranded DNA by the polymerase chain reaction,” Gene, 151:119-123 (1994).
Wondrak et al., “Influence of flanking sequences on the dimer stability of human immunodeficiency virus type 1 protease,” Biochemistry, 35:12957-12962 (1996).
Wu et al., “Structural basis for specificity of retroviral proteases,” Biochemistry, 37:4518-4526 (1998).

### BACKGROUND OF THE INVENTION

1. Field of the Invention

The present invention relates generally to arrays and methods for the parallel detection and analysis of up to a large number of proteins in a sample. More specifically, the present invention relates to proteomics and the measurement of gene activity at the protein level in cells. It also can be related to the detection of viruses, bacteria (pathogenic agents) and bio-warfare agents.

2. Description of Related Art

Although attempts to evaluate gene activity and to decipher biological processes including those of disease processes and drug effects have traditionally focused on genomics, proteomics offers a more direct and promising look at the biological functions of a cell. Proteomics involves the qualitative and quantitative measurement of gene activity by detecting and quantitating expression at the protein level, rather than at the messenger RNA level. Proteomics also involves the study of non-genome encoded events including the post-translational modification of proteins, interactions between proteins, and the location of proteins within the cell. The structure, function, or levels of activity of the proteins expressed by a cell are also of interest. Essentially, proteomics involves the study of part or all of the status of the total protein contained within or secreted by a cell.

The study of gene expression at the protein level is important because many of the most important cellular processes are regulated by the protein status of the cell, not by the status of gene expression. Also, the protein content of a cell is highly relevant to drug discovery efforts since most drugs are designed to be active against protein targets.

Measuring the mRNA abundances of a cell provides only an indirect and incomplete assessment of the protein content of a cell. The level of active protein that is produced in a cell is often determined by factors other than the amount of mRNA produced. For instance, both protein maturation and protein degradation are actively controlled in the cell and a protein's activity status can be regulated by post-translational modifications. Furthermore, the extreme liability of RNA in samples due to chemical and enzymatic degradation makes the evaluation of genetic expression at the protein level more practical than at the mRNA level.

Current technologies for the analysis of proteomes are based on a variety of protein separation techniques followed by identification of the separated proteins. The most popular method is based on 2D-gel electrophoresis followed by “in-gel” proteolytic digestion and mass spectroscopy. This technique is especially difficult for large proteins, proteins that are difficult to digest and to protein mixtures. Although the above described methods can provide structural protein information, they cannot provide functional information, for example, protein-protein interaction, affinity constants of such interaction, that are extremely important to drug discovery—i.e., development of drugs that promote or block specific protein function-interaction. Alternatively, Edman methods may be used for the sequencing. This 2D-gel technique requires large sample sizes, is time consuming, and is currently limited in its ability to reproducibly resolve a significant fraction of the proteins expressed by a human cell. Techniques involving some large-format 2D-gels can produce gels which separate a larger number of proteins than traditional 2D-gel techniques, but reproducibility is still poor and over 95% of the spots cannot be sequenced due to limitations with respect to sensitivity of the available sequencing techniques. The electrophoretic techniques are also plagued by a bias towards proteins of high abundance.

Standard assays for the presence of an analyte in a solution, such as those commonly used for diagnostics, for instance, involve the use of an antibody which has been raised against the targeted antigen. Multianalyte assays known in the art involve the use of multiple antibodies and are directed towards assaying for multiple analytes. However, these multianalyte assays have not been directed towards assaying the total or partial protein content of a cell or cell population. Furthermore, sample sizes required to adapt such standard antibody assay approaches to the analysis of even a fraction of the estimated 100,000 or more different proteins of a human cell and their various modified states are prohibitively large. Automation and/or miniaturization of antibody assays are required if large numbers of proteins are to be assayed simultaneously. Materials, surface coatings, and detection methods used for macroscopic immunoassays and affinity purification are not readily transferable to the formation or fabrication of miniaturized protein arrays. Miniaturized DNA chip technologies have been developed (for example, see U.S. Pat. Nos. 5,412,087, 5,445,934, and 5,744,305) and are currently being exploited for the screening of gene expression at the mRNA level. These chips can be used to determine which genes are expressed by different types of cells and in response to different conditions. However to date, DNA biochip technology has not been transferable to protein-binding assays such as antibody assays because the chemistries and materials used for DNA biochips have not been readily transferable to use with proteins. Nucleic acids such as DNA withstand temperatures up to 100° C., can be dried and re-hydrated without loss of activity, and can be bound physically or chemically directly to organic adhesion layers supported by materials such as glass while maintaining their activity. In contrast, proteins such as antibodies are preferably kept hydrated and at ambient temperatures are sensitive to the physical and chemical properties of the support materials. Therefore, maintaining protein activity at the liquid-solid interface requires entirely different immobilization strategies-than those used for nucleic acids. The proper orientation of the antibody or other protein at the interface is desirable to ensure accessibility of their active sites with interacting molecules. With miniaturization of the chip and decreased feature sizes, the ratio of accessible to non-accessible and the ratio of active to inactive antibodies or proteins become increasingly relevant and important.

U.S. Pat. Nos. 6,329,209 and 6,365,418 disclose arrays of protein capture agents that include protein immobilization regions, each of which contains a hydrophobic monolayer, a hydrophilic monolayer and functional groups attached to the hydrophobic/hydrophilic monolayers, wherein the hydrophobic and hydrophilic monolayers resist non-specific protein binding.

The techniques described in U.S. Pat. Nos. 6,329,209 and 6,365,418 have limitations in formation of the protein array. For example, the size of each protein-binding region on the array surface depends on droplet size. This reduces the number of array elements that can be contained on the surface. Possible denaturation of protein-capture agents during droplet drying limits the number of array elements. Other problems related to protein-capture hydrodynamic characteristics (like viscosity) limit this technique to the formation of protein arrays. The present invention is advantageous as compared to existing protein chips, including those discussed in the patents mentioned above, in that activation (immobilization of the proteins) of the inventive micro-array surface is simultaneous for all array pixels (patches) in the native (buffer) environment. This saves time, saves money on expensive robotic equipment, and allows use of any protein for surface activation, not only robust proteins such as antibodies. The system flexibility thus increases dramatically. Creation of any custom protein array at the surface, on site where the protein expression analysis or bio-warfare agent detection is needed is thus possible. There thus remains a need for the ability to assay in parallel a multitude of proteins expressed by a cell or a population of cells in an organism, including up to the total set of proteins expressed by the cell or cells efficiently and in a cost-effective manner.

Other publication, for example Niemeyer et al. ((1994) Nucleic Acid Research 22(25): 5530-5539), do not take into consideration strong nonspecific binding of the protein to the biochip surface. Careful choice of the type of molecules, that fill gaps between the instigator specific molecules on the template surface, significantly decreases nonspecific binding of the proteins.

### SUMMARY OF THE INVENTION

The present invention is directed to arrays and methods of use thereof that satisfy the need to assay in parallel a multitude of proteins expressed by a cell or population of cells in an organism, including up to the total protein content of a cell or for detecting pathogen i.e., agents including viruses and bacteria.

In accordance with the present invention, a template-containing an array of specific binding regions made from small robust molecules specific to a determined instigator are provided. The present invention permits the carrying out of protein chip activation in a flow cell, or in a non flow cell apparatus wherein solutions of specific instigators are added in sequence or a mixture of all (or part) of the instigators are added. The instigators, each of which is specific to a part of a binding molecule (Y) at a single binding region of the array, ultimately forms a well-determined protein chip. In contrast to the protein-capture agents in the U.S. Pat. Nos. 6,329,209 and 6,365,418 patents, the binding molecules and instigators are not exposed to the air which can cause protein denaturation and deactivation. The size and shape of the array elements will be determined by the characteristics of the template.

Specifically for high throughput drug screening, the instigator can be linked to a potential drug molecule. Activation of the template surface with a plurality of the instigator-drug heterodimers will lead to formation of the biochip for drug screening.

The present invention allows the highest order of process automation for biochip preparation, modification, measurement and analysis.

The template of the protein array on the substrate surface can be fabricated using photolithography, micromolding, wet chemical or dry etching, laser ablation technique, inkjet, droplet techniques or any combination of such techniques.

The substrate can be polymers, glass, silicon or other materials that can be used as a support for a protein array. Preferable material is glass that is transparent in the visible spectral region or optical polymers. The surface of the substrate may have a coating, for example is gold film. The gold film can be covered with a self assembled monolayer (SAM), for example polyethylene oxide (PE) terminated SAM that resists protein adsorption and can be used as a mask for patches (pixels) of the array formation (FIG. 4). On the next step of template formation, a small spot of the gold surface covered with SAM equal to the patch (pixel) size of the array is etched using, e.g., the laser ablation technique to remove SAM from the gold surface. Finally, to form the first pixel (patch) of the template of the protein array, the spot that now contains a clean gold surface is exposed to the reagent (binding molecules) that forms a patch on the template surface that is able to bind a specific instigator. The reagent (binding molecules) can be the SAM terminated with DNA, PNA or others (FIGS. 5 and 6). To enhance the binding of the instigator to the patch, the surface concentration of the reagent (binding molecules) should be carefully chosen. This can be done using molecular engineering techniques. To form a whole template, the procedure should be repeated for every template pixel beginning with the laser ablation step, but at the end applying different reagent (binding molecules) to bind different instigators (FIG. 4).

As discussed above, other techniques based on the inkjet or the droplet technique can be used for template formation. For example, very small volumes of the liquid (nano-liters or pico-liters) containing reagents (binding molecules) that specifically bind the instigator can be applied to the substrate surface containing a bare gold coating using spray by the inkjet technique or the droplet technique. Every drop or spray contains reagents specific to different instigators. After the binding of a plurality of the binding molecules with the gold surface, the patches of the template of the protein array will be ready to bind a plurality of instigators in a specific manner. To prevent nonspecific protein adsorption, the surface of the bare gold in between the patches can be filled out with PE terminated SAM.

Other techniques can be based on the combination of the above described techniques with modification of the glass surface without gold coating. One example is the well-known Affymetrix DNA chip, which itself can be used as a template for protein array formation.

In one embodiment, the present invention provides an array comprising a substrate, including a template of chemically robust molecules (“binding molecules”) immobilized on the substrate, wherein each binding molecule is capable of binding an instigator, which in turn binds a particular expression product, or a fragment thereof, of a cell or population of cells in an organism.

In a second embodiment, the invention provides an array of bound proteins which comprises the array of binding molecules of the invention having respective instigators attached thereto and a plurality of different proteins which are expression products, or fragments thereof, of a cell or population of cells in an organism attached to the instigators. Each of the different proteins is bound to a different binding molecule on the array via its respective instigator.

Methods of using the arrays of binding molecule and instigators of the invention are also provided. In one embodiment of the invention, a method of assaying in parallel for a plurality of different proteins in a sample which are expression products, or fragments thereof, of a cell or a population of cells in an organism, is provided which comprises first delivering the sample to the array of binding molecule of the invention under conditions suitable for protein binding, wherein each of the proteins being assayed is coupled to an instigator that, in turn, is a binding partner of at least one binding molecule on the array. The final step comprises detecting, either directly or indirectly, for the presence or amount of protein bound to each instigator on the array. This method optionally further comprises the step of further characterizing the proteins bound to at least one instigator on the array.

In another embodiment of the invention, a method for determining the protein expression pattern of a cell or a population of cells in an organism is provided which comprises first delivering a sample containing the expression products, or fragments thereof, of the cell or population of cells to the array of binding molecules/instigators of the invention under conditions suitable for protein binding. The final step comprises detecting, either directly or indirectly, for the presence or amount of protein bound to each binding molecule/instigator on the array. In an alternative embodiment, a similar method for comparing the protein expression patterns of two cells or populations of cells is also provided.

In still another embodiment of the invention, an alternative method of assaying in parallel for a plurality of different proteins in a sample that are expression products, or fragments thereof, of a cell or a population of cells in an organism is provided. The method of this embodiment comprises first contacting the sample with an array of spatially distinct and different binding molecules/instigators, wherein each of the proteins being assayed is a binding partner of at least one of the instigators. The last step of the method involves detecting, either directly or indirectly, for the presence or amount of protein bound to each of the instigators on the array.

In a still further embodiment, a method of producing an array of binding molecules/instigators is provided which comprises the following steps: selecting binding molecules/instigators, wherein the binding molecules/instigators are selected by their binding affinity to one another, wherein the instigator is further selected by its ability to bind proteins from a cellular extract or body fluid; producing a plurality of binding molecules; immobilizing the binding molecules on the substrate surface to form a plurality of binding molecules on discrete, known regions of the surface of a substrate; and adding respective instigators that attach to the binding molecules.

In still a further embodiment, the technique can be used for detection of viruses, bacteria or other pathogens in biological fluids.

In still a further embodiment, the technique can be used for detection of viruses, bacteria or other pathogens in air or water samples in a combat or a non combat environment.

In still a further embodiment, the technique can be used for detection of bacteria secreted proteins.

### BRIEF DESCRIPTION OF THE FIGURES

FIG. 1 shows an example of the template of the invention, designed on a solid substrate with active spots (i.e., pixels or bio-array elements) ready to bind instigators.

FIG. 2 shows an example of the instigator of the invention. End #1 is ready to bind to a complimentary site on the template.

FIG. 3 shows an example of the bioarray of the invention. Wherein the template is activated for proteomics applications.

FIG. 4 shows an example of the biochip template formation using laser ablation technique.

FIG. 5 shows an example of the binding molecules attached to the glass (1) substrate covered with gold film (2) through adhesion chromium layer (3).

FIG. 6. shows an example of the binding molecules attached to the glass (1) substrate.

FIG. 7. shows an example of the formation of the instigator

### DETAILED DESCRIPTION OF THE INVENTION

A variety of arrays and methods useful for multianalyte analyses and analyses of protein expression and modification in cells are provided by the present invention.

Definitions

The term “protein-binding moiety” means a molecule or a multi-molecular complex, which can bind a protein to itself. The protein-binding moiety will most typically be a biomolecule such as a protein or a polynucleotide. The biomolecule may optionally be a naturally occurring, recombinant, or synthetic biomolecule. Antibodies or antibody fragments are highly suitable as protein-binding moieties. Antigens may also serve as protein-binding moieties, since they are capable of binding antibodies. Receptors which bind protein ligands are another example of a possible protein-binding moieties. For instance, protein-binding moiety are understood not to be limited to agents which only interact with their binding partners through noncovalent interactions. Protein-binding moieties may also optionally become covalently attached to proteins which they bind. For instance, the protein-binding moiety may be photocrosslinked to its binding partner following binding.

The term “binding partner” means a protein which is bound by a particular protein-binding moiety, preferably in a substantially specific manner. In some cases, the protein-binding moiety may be a cellular or extracellular protein and the binding partner may be the entity normally bound in vivo. In other embodiments, however, the binding partner may be the protein or peptide on which the protein-binding moiety was selected (through in vitro or in vivo selection) or raised (as in the case of antibodies). A binding partner may be shared by more than one protein-binding moiety. For instance, a binding partner which is bound by a variety of polyclonal antibodies may bear a number of different epitopes. One protein-binding moiety may also bind to a multitude of binding partners, for instance, if the binding partners share the same epitope. The binding partner may be viruses, bacteria or other pathogens or products of the bacteria, e.g., secreted proteins.

A “protein” means a polymer of amino acid residues linked together by peptide bonds. The term, as used herein, refers to proteins, polypeptides, and peptides of any size, structure, or function. Typically, however, a protein will be at least six amino acids long. Preferably, if the protein is a short peptide, it will be at least about 10 amino acid residues long. A protein may be naturally occurring, recombinant, or synthetic, or any combination of these. A protein may also be just a fragment of a naturally occurring protein or peptide. A protein may be a single molecule or may be a multi-molecular complex. The term protein may also apply to amino acid polymers in which one or more amino acid residues is an artificial chemical analogue of a corresponding naturally occurring amino acid. An amino acid polymer in which one or more amino acid residues is an “unnatural” amino acid, not corresponding to any naturally occurring amino acid, is also encompassed by the use of the term “protein” herein.

A “fragment of a protein” means a protein, which is a portion of another protein. For instance, fragments of a protein may be polypeptides obtained by digesting full-length protein isolated from cultured cells. A fragment of a protein will typically comprise at least six amino acids. More typically, the fragment will comprise at least ten amino acids. Preferably, the fragment comprises at least about 16 amino acids.

An “expression product” is a biomolecule, such as a protein, which is produced when a gene in an organism is expressed. An expression product may optionally comprise post-translational modifications.

The term “antibody” means an immunoglobulin, whether natural or partially or wholly synthetically produced. All derivatives thereof which maintain specific binding ability are also included in the term. The term also covers any protein having a binding domain which is homologous or largely homologous to an immunoglobulin binding domain. These proteins may be derived from natural sources, or partly or wholly synthetically produced. An antibody may be monoclonal or polyclonal. The-antibody may be a member of any immunoglobulin class, including any of the human classes: IgG, IgM, IgA, IgD, and IgE.

The term “antibody fragment” refers to any derivative of an antibody which is less than full-length. Preferably, the antibody fragment retains at least a significant portion of the full-length antibody's specific binding ability. The antibody fragment may be produced by any means. For instance, the antibody fragment may be enzymatically or chemically produced by fragmentation of an intact antibody or it may be recombinantly produced from a gene encoding the partial antibody sequence. Alternatively, the antibody fragment may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

A “population of cells in an organism” means a collection of more than one cell in a single organism or more than one cell originally derived from a single organism. The cells in the collection are preferably all of the same type. They may all be from the same tissue in an organism, for instance. Most preferably, gene expression in all of the cells in the population is identical or nearly identical.

“Conditions suitable for protein binding” means those conditions (in terms of salt concentration, pH, detergent, protein concentration, temperature, etc.) which allow for binding to occur between an immobilized protein-capture agent and its binding partner in solution. Preferably, the conditions are not so lenient that a significant amount of nonspecific protein binding occurs.

A “body fluid” may be any liquid substance extracted, excreted, or secreted from an organism or tissue of an organism. The body fluid need not necessarily contain cells. Body fluids of relevance to the present invention include, but are not limited to, whole blood, serum, urine, plasma, cerebral spinal fluid, tears, sinovial fluid, and amniotic fluid. An “array” is an arrangement of entities in a pattern on a substrate. Although the pattern is typically a two-dimensional pattern, the pattern may also be a three-dimensional pattern.

A “binding molecule” is used herein to refer to a molecule that binds to a substrate and to an instigator.

A “binding region” means a discrete area of immobilized binding molecules on the surface of a substrate. The binding region may be of any geometric shape or may be irregularly shaped. For instance, the binding region may be, but need not necessarily be, square in shape.

“Proteomics” means the study of or the characterization of either the proteome or some fraction of the proteome. The “proteome” is the total collection of the intracellular proteins of a cell or population of cells and the proteins secreted by the cell or population of cells. This characterization most typically includes measurements of the presence, and usually quantity, of the proteins which have been expressed by a cell. The function, structural characteristics (such as post translational modification), and location within the cell of the proteins may also be studied. “Functional proteomics” refers to the study of the functional characteristics, activity level, and structural characteristics of the protein expression products of a cell or population of cells.

The term “substrate” refers to the bulk underlying, and core material of the arrays of the invention.

The term “fusion protein” refers to a protein composed of two or more polypeptides that, although typically unjoined in their native state, are joined by their respective amino and carboxyl termini through a peptide linkage to form a single continuous polypeptide. It is understood that the two or more polypeptide components can either be directly joined or indirectly joined through a peptide linker/spacer.

An “instigator” is a molecule or a complex of molecules that specifically binds to both the binding molecule and to the protein of interest.

The term “normal physiological condition” means conditions that are typical inside a living organism or a cell. While it is recognized that some organs or organisms provide extreme conditions, the intra-organismal and intra-cellular environment normally varies around pH 7 (i.e., from pH 6.5 to pH 7.5), contains water as the predominant solvent, and exists at a temperature above 0° C. and below 50° C. It will be recognized that the concentration of various salts depends on the organ, organism, cell, or cellular compartment used as a reference.

Arrays of the Invention

The present invention is directed to arrays of binding molecules which can bind a plurality of instigators, that in turn bind to proteins that are the expression products, or fragments thereof, of a cell or population of cells in an organism and therefore can be used to evaluate gene expression at the protein level. Typically, the arrays comprise micrometer or nanometer-scale, two-dimensional patterns of regions of binding molecules with or without instigators immobilized on the surface of the substrate.

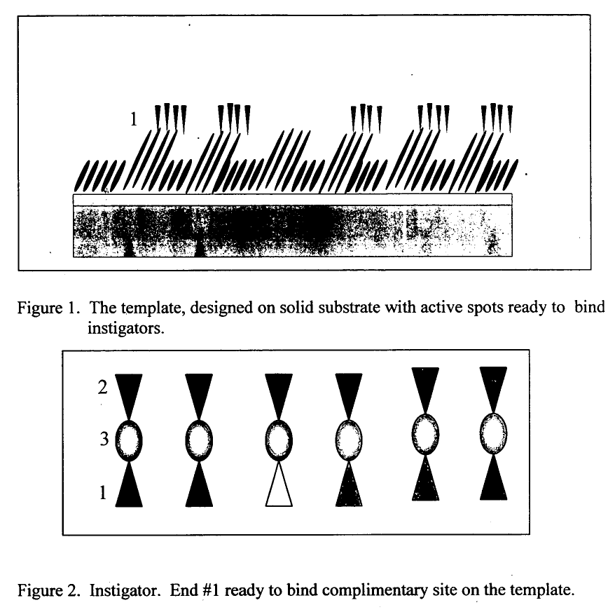

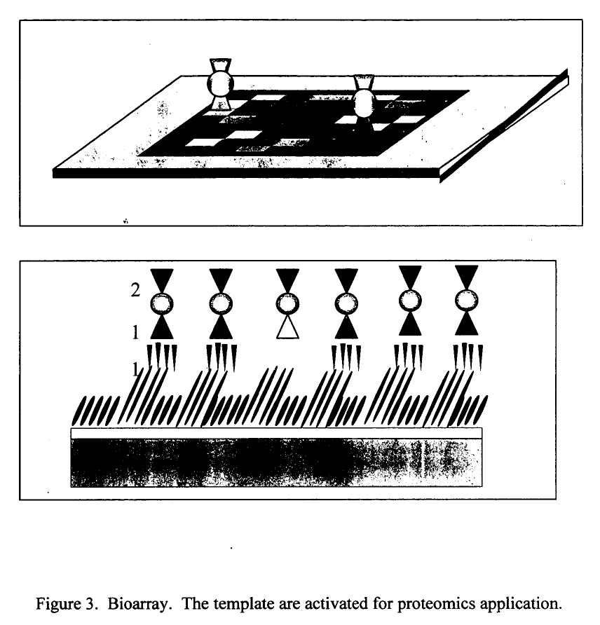

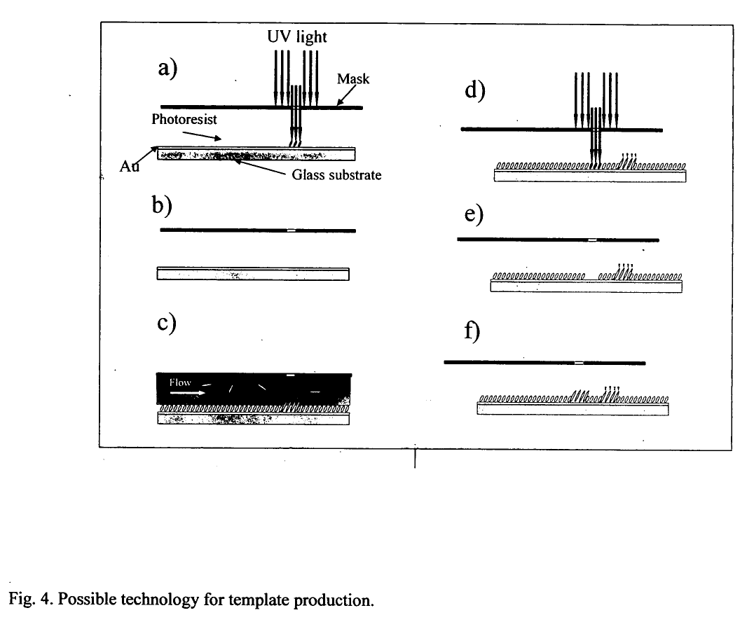

The two main elements of the invention are a template and an instigator (heterodimer). The template (FIG. 1) contains molecules compliment to #1 end of the heterodimer instigator (FIG. 2).

The template (FIG. 1) is formed on a substrate made from e.g., glass, plastic, silicon or other material covered with spots (pixels or bio-array elements) of small chemically robust molecules (FIG. 1, #1), e.g., single stranded DNA, PNA, polypeptides or any other molecules that have a complimentary binding pair (e.g., DNA-DNA pair or DNA-PNA pair, polypeptide-antibody, etc.). Between the spots, the surface may be covered with molecules that prevent nonspecific protein adsorption. The template can be made using standard techniques such as inkjet printing, photolithography, droplet techniques or others. For example, any DNA array can be used as a prototype for the template. Every single pixel of the array should be made using molecularly engineered techniques to enhance specific binding of the instigator.

The instigator (FIG. 2) (heterodimer) consists of two molecules. As discussed above, molecule #1 (FIG. 2) is a compliment to the molecule on the template surface (#1 on template and #1 instigator), another molecule #2 (FIG. 2) sets up the bio-array element for the protein screening or for the screening of small molecules—e.g., candidates for drug discovery.

The instigator can be made using a variety of methods. #2 depends on proteomics application and can be, e.g., an antibody, Fab fragments, a protein, or a drug candidate in high throughput drug screening.

The two molecules can be immobilized on beads, phospholipids vesicles or liposoms or the two molecules can be linked through avidin or strepavidin interaction with biotin attached to the molecules, through direct chemical binding, using PBA (phenyl boronic acid) and SHA (salicylhydroxamic acid), the two antibodies can be liked through the protein A, etc.

In one embodiment of the invention, the array and a plurality of regions arranged in discrete, known areas on portions of the substrate surface wherein each region comprises immobilized binding molecules, each of which is capable of binding a particular instigator, which, in turn, is capable of binding a particular expression product, or a fragment thereof, of a cell or population of cells in an organism e.g., bacteria, virus or secreted proteins of the bacteria.

The binding molecules of the template are preferably covalently immobilized on the regions of the array, either directly or indirectly. The instigators are preferably non-covalently immobilized on the specific patches of the array.

In most cases, the array will comprise at least about ten regions. In a preferred embodiment, the array comprises at least about 50 regions. In a particularly preferred embodiment the array comprises at least about 100 regions. In alternative preferred embodiments, the array of binding molecules may comprise more than 10³, 10⁴ or 10⁵ regions. Typically, only one type of binding molecule is present on a single patch of the array, but for specific applications, for example for bacteria or viruses detection, it can be multi-molecular binding.

The arrays of the invention can have any number of a plurality of different binding molecules. Typically the array comprises at least about ten different binding molecules. Preferably, the array comprises at least about 50 different binding molecules. More preferably, the array comprises at least about 100 different binding molecules. Alternative preferred arrays comprise more than about 10³ different binding molecules or more than about 10⁴ different binding molecules. The array may even optionally comprise more than about 10⁵ different binding molecules.

The number of different binding molecules on the array will vary depending on the application desired. For instance, if the array is to be used as a diagnostic tool in evaluating the status of a tumor or other diseased tissue in a patient, an array comprising less than about 100 different binding molecules (coupled to instigators) may suffice since the necessary binding partners of the binding molecules/instigators on the array are limited to only those proteins whose expression is known to be indicative of the disease condition. For the detection of the bacteria or viruses the number of the array elements can be small also. However, if the array is to be used to measure a significant portion of the total protein content of a cell, then the array preferably comprises at least about 10,000 different binding molecules/instigators. Alternatively, a more limited proteomics study, such as a study of the abundances of various human transcription factors, for instance, might only require an array of about 100 different binding molecules/instigators.

In one embodiment of the invention, the binding partners of the plurality of binding molecules/instigators on the array are proteins, which are all expression products, or fragments thereof, of a cell or population of cells of a single organism. The expression products may be proteins, including peptides, of any size or function. They may be intracellular proteins or extracellular proteins. The expression products may be from a one-celled or multicellular organism. The organism may be a plant or an animal. In a preferred embodiment of the invention, the binding partners are human expression products, or fragments thereof.

In one embodiment of the invention, the binding partners of the binding molecules/instigators of the array may be a randomly chosen subset of all the proteins, including peptides, which are expressed by a cell or population of cells in a given organism or a subset of all the fragments of those proteins. Thus, the binding partners of the binding molecules/instigators of the array optionally represent a wide distribution of different proteins from a single organism.

In an alternative embodiment of the invention, the proteins, which are the binding partners of the binding molecules/instigators of the array, may be fragments of the expression products of a cell or population of cells in an organism.

A first portion of the instigator is capable of binding to the binding molecule. A second portion of the instigator can be any molecule or complex of molecules, which has the ability to bind a protein and immobilize it to the site of the binding molecule on the array. Preferably, the second portion binds its binding partner in a substantially specific manner. Hence, the second portion may optionally be a protein whose natural function in a cell is to specifically bind another protein, such as an antibody or a receptor. Alternatively, the second portion may instead be a partially or wholly synthetic or recombinant protein, which specifically binds a protein.

In a preferred embodiment of the array, however, the second portion is a protein. In a particularly preferred embodiment, the second portion is an antibody or an antibody fragment. Although antibody moieties are exemplified herein, it is understood that the present arrays and methods may be advantageously employed with other agents capable of binding to protein.

Upon using the array of binding molecules/instigators to bind a plurality of expression products, or fragments thereof, an array of bound proteins is created. Thus, another embodiment of the invention provides an array of bound proteins which comprises (a) a binding molecule/instigator array of the invention and (b) a plurality of different proteins which are expression products, or fragments thereof, of a cell or a population of cells in an organism, wherein each of the different proteins is bound to a binding molecule/instigator on a separate region of the array. Preferably, each of the different proteins is non-covalently bound.

Substrates and Templates

The substrate of the array may be either organic or inorganic, biological or non-biological, or any combination of these materials. In one embodiment, the substrate is transparent or translucent. The portion of the surface of the substrate on which the regions reside is preferably flat and firm or semi-firm. However, the array of the present invention need not necessarily be flat or entirely two-dimensional.

Numerous materials are suitable for use as a substrate in the array embodiment of the invention. For instance, the substrate of the invention array can comprise a material selected from a group consisting of silicon, silica, quartz, glass, controlled pore glass, carbon, alumina, titania, tantalum oxide, germanium, silicon nitride, zeolites, and gallium arsenide. Many metals such as gold, platinum, aluminum, copper, titanium, and their alloys are also options for substrates of the array. In addition, many ceramics and polymers may also be used as substrates. Polymers which may be used as substrates include, but are not limited to, the following: polystyrene; poly(tetra)fluoroethylene (PTFE); polyvinylidenedifluoride; polycarbonate; polymethylmethacrylate; polyvinylethylene; polyethyleneimine; poly(etherether)ketone; polyoxymethylene (POM); polyvinylphenol; polylactides; polymethacrylimide (PMI); polyalkenesulfone (PAS); polypropylethylene, polyethylene; polyhydroxyethylmethacrylate (HEMA); polydimethylsiloxane; polyacrylamide; polyimide; and block-copolymers. Preferred substrates for the array include silicon, silica, glass, and polymers. The substrate on which the binding molecules reside may also be a combination of any of the aforementioned substrate materials.

An array of the present invention may optionally further comprise a coating between the substrate and the binding molecules. This coating may either be formed on the substrate or applied to the substrate. The substrate can be modified with a coating by using thin-film technology based, for instance, on physical vapor deposition (PVD), plasma-enhanced chemical vapor deposition (PECVD), or thermal processing. Alternatively, plasma exposure can be used to directly activate or alter the substrate and create a coating. For instance, plasma etch procedures can be used to oxidize a polymeric surface (for example, polystyrene or polyethylene to expose polar functionalities such as hydroxyls, carboxylic acids, aldehydes and the like) which then acts as a coating.

A plurality of regions, each of which is made from a different chemical entity, for example, an Affymetrix DNA chip, wherein every region has a different DNA molecule, is the point wherein the binding molecules reside.

The regions comprise binding molecules which in turn comprise the formula (X)a-R-(Y)b, where b is equal to the number of regions on the surface (number of the array elements), and Yb specifically non-covalently binds a specific instigator.

In a preferred embodiment, each of the regions comprises a monolayer of binding molecules of the formula (X)a-R-(Y)b and the regions are separated with monolyaer that resists to non-specific adsorption of protein instigators.

(Y)b specifically is a plurality of functional groups that specifically non-covalently binds a specific instigator.

(Y)b can be any functional group that can specifically non-covalently or covalently bind a specific instigator including polynucleotides, polypeptides, PNA, Ab, Fab, etc.

All individual regions are mixed self-assembled monolayers of nonspecific and specific Y groups.

The portion of the binding molecule that binds to the surface will be the same (e.g., —S— or —O—Si—O). The other portion of the binding molecule will be a plurality of small molecules (polynucleotides, polypeptides) that can bind other molecules complimentary (for example, DNA-DNA, polypeptide—antibody, PNA-DNA).

The component, Y, is a functional group responsible for binding an instigator. In a preferred embodiment of the invention, the Y group is either highly reactive (activated) towards the instigator or is easily converted into such an activated form. In a preferred embodiment, the coupling of Y with the instigator occurs readily under normal physiological conditions not detrimental to the ability of the instigator to bind its binding partner. The functional group Y may either form a covalent linkage or a noncovalent linkage with the instigator. In a preferred embodiment, the functional group Y forms a non-covalent high affinity linkage with the instigator. It is understood that following the attachment of the instigator to Y, the chemical nature of Y may have changed.

After completion of formation of the regions (patches, pixels, spots, array elements), the instigators may be attached to the regions via interaction with the Y-functional group. Y-functional groups, which fail to react with any instigators, if necessary can be quenched prior to use of the array. For quenching can be used part #1 of the instigator (FIG. 2) terminated with polyethylene oxide or other group that prevents nonspecific binding of the proteins.

The instigators function to enhance immobilization of the protein, and to direct the protein to the specific region on the surface during template activation. The binding molecules and instigators used on the array may be produced by any of the variety of means known to those of ordinary skill in the art.

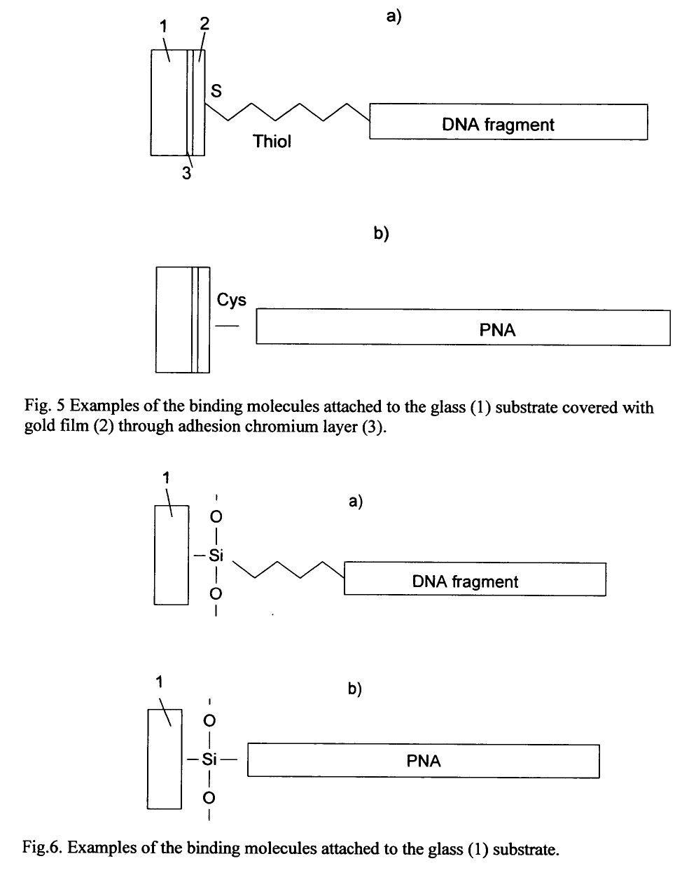

FIG. 5 shows one example of the binding molecule. In this example the substrate comprises glass covered with gold film. a) The binding molecule with DNA fragment that can contain 20 or more bases attached to the gold surface through the short (usually 6 methylene groups) thiol linker. b) binding molecule based on the PNA can be linked to the gold surface through the cystein amino acid or other amino acids that contain sulfur bond active to the gold surface.

FIG. 6 shows another example of the binding molecule. In this example the substrate is a glass surface. The binding molecules in this case can be attached to the surface through—silane groups, for example dialcosilane.

These are preferred examples for the binding molecules and the chemistry of the attachment of these molecules to the surface. Another example of binding molecules can be polypeptides or any other simple robust molecules that exhibit a combination of elements to form a plurality of the specific binding sites on the surface. In some cases the binding molecules can be proteins or fragments of the proteins, for example Fab fragments of antibodies or combinations of proteins with small molecules.

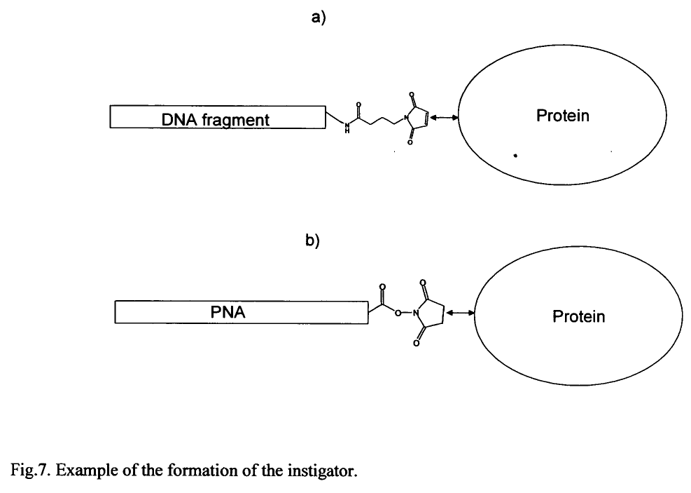

The instigator is a heterodimer that consists of two functional groups (FIG. 7). A first group binds the instigator to the surface; a second group activates the protein array. In most cases, the second group is a protein. These two groups can be linked using many functional groups. Possibilities for these types of functional groups include, but are not limited to, simple moieties such as a hydroxyl, carboxyl, amino, aldehyde, carbonyl, methyl, methylene, alkene, alkyne, carbonate, aryliodide, or a vinyl group. Appropriate modes of activation would be obvious to one skilled in the art. Alternatively, it can comprise a functional group that requires photoactivation prior to becoming activated enough to bind the second group (protein) to form the instigator. One of the examples to form the instigator is shown in FIG. 7. DNA fragment or PNA can be linked to the protein through N-hydroxysuccimide or maleimedyl functional group. There are many other methods that can be used to make the instigator. In the case when binding molecule (at the surface) is polypeptide instigator can be made using antibodies linked through Fc end using protein A. For example, the instigator can be formed using phospholipid vesicles, polymer beads, biotin-avidin (streptavidin) chemistry, using phenylboronic acid (PBA) and salicylhydroxamic asid (SHA) complex formation and many other types of binding.

Uses of the Arrays

The present invention also provides methods of using the inventive arrays. In general, for a variety of applications including proteomics, diagnostics and pathogen detection, the methods of the invention involve the delivery of sample containing proteins to be analyzed to the arrays. After the proteins of the sample have been allowed to interact with and become immobilized on the regions of the array comprising binding molecules/instigators with the appropriate specificity, the presence and/or amount of protein bound at each region is then determined.

Use of one of the binding molecule/instigator arrays of the invention may optionally involve placing the two-dimensional array in a flowchamber. The cover over the array in the flowchamber is preferably transparent or translucent and may be part of a detection system that monitors interaction between the instigators immobilized on the array and protein in a solution such as a cellular extract.

The sample which is delivered to the array will typically be a fluid. In a preferred embodiment of the invention, the sample is a cellular extract or a body fluid. The sample to be assayed may optionally comprise a complex mixture of proteins, including a multitude of proteins which are not binding partners of the instigators of the array. If the proteins to be analyzed in the sample are membrane proteins, then those proteins will typically need to be solubilized prior to administration of the sample to the array. If the proteins to be assayed in the sample are proteins secreted by a population of cells in an organism, a sample which is derived from a body fluid is preferred. If the proteins to be assayed in the sample are intracellular, a sample which is a cellular extract is preferred. In one embodiment of the invention, the array may comprise instigators which bind fragments of the expression products of a cell or population of cells in an organism. In such a case, the proteins in the sample to be assayed may have been prepared by performing a digest of the protein in a cellular extract or a body fluid. In an alternative application of the array, the proteins from only specific fractions of a cell are collected for analysis in the sample.

A wide range of detection methods are applicable to the methods of the invention. As desired, detection may be either quantitative or qualitative. The inventive array can be interfaced with optical detection methods such as absorption in the visible or infrared range, chemiluminescence, and fluorescence (including lifetime, polarization, fluorescence correlation spectroscopy (FCS), and fluorescencce-resonance energy transfer (FRET)). Other modes of detection, including surface plasmon resonance, are compatible with many embodiments of the invention.

The arrays of the present invention are particularly useful for proteomics. Those arrays which comprise significant numbers of binding molecules/instigators of different specificity on separate regions can bind significant numbers of proteins which are expression products, or fragments thereof, of a cell or population of cells in an organism and are particularly suitable for use in applications involving proteomics. For instance, an array with at least about 10³ and up to about 10⁵ different binding molecules/instigators can provide a highly comprehensive picture of the protein content of the cell under a specific set of conditions.

In one embodiment of the invention, a method of assaying in parallel for a plurality of different proteins in a sample which are expression products, or fragments thereof, of a cell or a population of cells in an organism, is provided which comprises the following steps: first, delivering the sample to an array of spatially distinct regions of different binding molecules/instigators under conditions suitable for protein binding, wherein each of the proteins being assayed is a binding partner of a second portion of an instigator of at least one region on the array; next, optionally washing said array to remove unbound or nonspecifically bound components of the sample from the array; and in a final step, detecting, either directly or indirectly, for the presence or amount of protein bound to each region of the array.

In another embodiment of the invention, a method of assaying in parallel for a plurality of different proteins in a sample which are expression products, or fragments thereof, of a cell or a population of cells in an organism, comprises first delivering the sample to the inventive array under conditions suitable for protein binding, wherein each of the proteins being assayed is a binding partner of the second portion of at least one instigator on the array. The first step may be followed by an optional step of washing the array with fluid to remove unbound or nonspecifically bound components of the sample from the array. Lastly, the presence or amount of protein bound to each region is detected, either directly or indirectly.

The methods of assaying in parallel for a plurality of different proteins in a sample which are expression products, or fragments thereof, of a cell or a population of cells in an organism, optionally comprise the additional step of further characterizing the protein bound to at least one region of the array. This step is typically designed to identify the nature of the protein bound to the protein-capture agent of a particular patch. In some cases, the entire identity of the bound protein may not be known and the purpose of the further characterization may be the initial identification of the mass, sequence, structure and/or activity of the bound protein. In other cases, the basic identity of the protein may be known, but the post-translational modification, activation state, or some other feature of the protein may not be known. In one embodiment, the step of further characterizing the proteins involves measuring the activity of the proteins.

In another embodiment, the present invention provides a method of determining the protein expression pattern of a cell or population of cells in an organism. This method involves first delivering a sample containing expression products, or fragments thereof, of the cell or population of cells to the array of the invention under conditions suitable for protein binding. The presence and/or amount of protein bound to each region can then be determined by a suitable detection means.

The arrays may also be used to compare the protein expression patterns of two cells or populations of cells. In this method, a sample containing expression products, or fragments thereof, of a first cell or population of cells is delivered to the inventive array of under conditions suitable for protein binding. In an analogous manner, a sample containing expression products, or fragments thereof, of a second cell or population of cells is delivered to a second array which is identical to the first array. Preferably, both arrays are then washed to remove unbound or nonspecifically bound components of the sample from the arrays. In a final step, the amounts of protein remaining bound to the regions of the first array are compared to the amounts of protein remaining bound to the corresponding regions of the second array. If it is desired to determine the differential protein expression pattern of two cells or populations of cells, for instance, then the amount of protein bound to the regions of the first array may be subtracted from the amount of protein bound to the corresponding regions of the second array.

Methods of comparing the protein expression of two cells or populations of cells are particularly useful for the understanding of biological processes. For instance, using these methods, the protein expression patterns of identical cells or closely related cells exposed to different conditions can be compared. Most typically, the protein content of one cell or population of cells is compared to the protein content of a control cell or population of cells. For instance, in one embodiment of the invention, one of the cells or populations of cells is neoplastic and the other cell is not. In another embodiment, one of the two cells or populations of cells being assayed is infected with a pathogen. Alternatively, one of the two cells or populations of cells has been exposed to a stressor and the other cell or population of cells serves as a control. The stressor may optionally be chemical, environmental, or thermal. One of the two cells may optionally be exposed to a drug or a potential drug and its protein expression pattern compared to a control cell.

Such methods of assaying differential gene expression at the protein level are useful in the identification and validation of new potential drug targets as well as for drug screening. For instance, the method may be used to identify a protein which is overexpressed in tumor cells, but not in normal cells. This protein may be a target for drug intervention. Inhibitors to the action of the overexpressed protein can then be developed. Alternatively, antisense strategies to inhibit the overexpression may be developed. In another instance, the protein expression pattern of a cell, or population of cells, which has been exposed to a drug or potential drug can be compared to that of a cell, or population of cells, which has not been exposed to the drug. This comparison will provide insight as to whether or not the drug has had the desired effect on a target protein (drug efficacy) and whether other proteins of the cell, or population of cells, have also been affected (drug specificity).

The arrays of the present invention are also suitable for diagnostic applications and suitable for use in diagnostic devices. The high density of the binding molecules/instigators on some arrays of the present invention enables a large number of different diagnostic tests to be formatted onto a single biochip. The inventive array can be used to evaluate the status of a disease condition in a tissue, such as a tumor, where the expression levels of certain proteins in the cells of the tissue is known to be indicative of a particular type of disease condition or stage of a disease condition. If certain patterns of protein expression are not previously known to be indicative of a disease state, the arrays of the invention can then first be used to establish this information.

Accordingly, in one embodiment, the invention provides a method of evaluating a disease condition in a tissue of an organism comprising first contacting the inventive array of with a sample comprising the expression products, or fragments thereof of the cells of the tissue being evaluated, wherein the contacting occurs under conditions suitable for protein binding and wherein the binding partners of a plurality of instigators on the array include proteins which are expression products, or fragment thereof, of the cells of the tissue and whose expression levels are indicative of the disease condition. The method next comprises detecting, either directly or indirectly, for the presence of protein to each region. In a preferred embodiment, the method further comprises the step of washing the array to remove unbound or nonspecifically bound components of the sample from the array. In such a method, the array will typically bind those proteins whose presence, absence, or relative amount in cells is known to be indicative of a particular type of disease condition or state of a disease condition.

### EXAMPLE

This examples is related to the detection of bacteria and viruses for bio-warfare applications. In this case, the array can contain a small number (it is known about 100 bio-warfare pathogens) of active patches. Every patch of the template in this case can be made using single stranded DNA molecules that compliment the single stranded DNAs of the instigators. These (last) DNA, which are linked to the antibodies specific to viruses or bacteria or to the transmembrane proteins or other proteins are able to specifically recognize pathogens using in bio-warfare. Made in this way, instigators are applied to the template surface and activate the array before measurement of the sample that can contain bio-warfare agents. After measurement the array can be reactivated. For example, heating of the sample will destroy DNA-DNA interaction and washing will remove all instigators and pathogens that were bound to the array. After this procedure, template of the array will be ready for the next measurement. The number of instigators on the template surface specific to the particular bio-warfare agent can be varied on the flight to enhance sensitivity and reliability of the measurements.

The following specific example is intended to illustrate the invention and should not be construed as limiting the scope of the claims:

The two main elements of the invention are a template and an instigator (heterodimer). The template (FIG. 1) contains molecules compliment to #1 end of the heterodimer (FIG. 2).

The template (FIG. 1) is formed on a substrate made from, e.g., glass, plastic, silicon or other material covered with spots (pixels or bio-array elements) of small chemically robust molecules (FIG. 1, #1), e.g., single stranded DNA, PNA, polypeptides or any other molecules that have a complimentary binding pair (e.g., DNA-DNA pair or DNA-PNA pair, polypeptide -antibody, etc.). Between the spots, the surface is covered with molecules that prevent nonspecific protein adsorption. The template can be made using standard techniques such as inkjet printing, photolithography, droplet techniques or others. For example, any DNA array can be used as a prototype for the template.

The instigator (FIG. 2) (heterodimer) consists of two molecules. As was discussed above, molecule #1 (FIG. 2) is a compliment to the molecule on the template surface (#1 on template and #1 instigator), another molecule #2 (FIG. 2) sets up the bio-array element for the protein screening or for the screening small molecules—e.g., candidates for drug discovery.

The instigator can be made using variety of the methods. #2 depends on proteomics application and can be, e.g., an antibody, Fab fragments, a protein, or a molecule candidate for a drug in high throughput drug screening.

The two molecules can be immobilized on the beads, the two molecules can be linked through avidin or strepavidin interaction with biotin attached to the molecules, or through direct chemical binding, using PBA (phenyl boronic acid) and SHA (salicylhydroxamic acid).

All documents cited in the above specification are herein incorporated by reference. Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in the art, are intended to be within the scope of the following claims.

1. An array device comprising:
a substrate having a surface;
one or more binding molecules on said substrate surface; and
one or more instigators, said one or more instigators each comprising (a) a single stranded DNA or RNA or PNA compliment to one or more of said binding molecules and (b) a protein-binding moiety, said protein-binding moiety is specific for a protein.

2. The device of claim 1 wherein at least two of said binding molecules are adjacent each other.

3. The device of claim 2 wherein at least two of said binding molecules are separated from each other by one or more border regions.

4. The device of claim 3 wherein said border regions are formed from non-binding molecules that prevent non-specific adsorption.

5. The device of claim 3 wherein said non-binding molecules are hydrophobic.

6. The device of claim 3 wherein said non-binding molecules are hydrophilic.
